(19) United States
(12) Patent Application Publication
(10) Pub. No.: US 2004/0137422 A1
(43) Pub. Date: Jul. 15, 2004

(54) Urine preservative tube

(76) Inventors: Robert S. Golabek JR., Towaco, NJ (US); Jack Mehl, Bradenton, FL (US); Jayraj Desai, Mumbai (IN)

Correspondence Address: Davie W. Highet, Becton, Dickinson and Company, 1 Becton Drive, Franklin Lakes, NJ, 07417, US

(21) Appl. No.: 10/685,198

(22) Filed: Oct. 14, 2003

Related U.S. Application Data
(60) Provisional application No. 60/439,178, filed on Jan. 10, 2003.

(51) Int. Cl. C12Q 1/00

(52) U.S. Cl. 435/4

(57) ABSTRACT

A urine stabilizing compound particularly suitable for plastic collection containers is provided. In one embodiment, the tube is formed by steps including providing a container, providing a formulation comprising mannitol, boric acid, sodium formate, sodium borate, water, glutamine, and a surfactant, and disposing the formulation into the container.

### RELATED APPLICATIONS

This application claims the benefit of U.S. Provisional Application No. 60/439,178 filed Jan. 10, 2003.

### BACKGROUND OF THE INVENTION

1. Field of the Invention

The invention relates to urine collection tubes that provide for preservation of the urine sample.

2. Discussion of the Related Art

Urine specimens are used for a variety of analytical tests. Urine, however, has the capability to support proliferation of bacteria, which may lead to incorrect results in any subsequent testing. Thus, it has been recognized that urine specimens need either special treatment, e.g., culturing within a short time of collection, refrigeration subsequent to collection, or stabilizing compounds therein.

Stabilizing formulations were a desired solution, since they avoided the need for quick culturing, or for refrigeration. Examples of such stabilizing compounds are reflected in U.S. Pat. Nos. 4,768,653, 4,336,880, and 4,258,032, the disclosures of which are hereby incorporated by reference.

However, such formulations were developed primarily for use in glass tubes. As the industry converts from glass to plastic, these formulations may not work as well, or may not work at all. For example, while glass has excellent gas and moisture barrier properties, plastics vary in their properties, particularly in moisture retention/transmission properties. Thus, liquid additives that might be suitable for glass tubes are not necessarily suitable for plastic tubes.

Thus, there is a need for a urine stabilizing formulation suitable for use in plastic collection tubes.

### SUMMARY OF THE INVENTION

The invention relates to a urine stabilizing compound particularly suitable for plastic collection containers. In one embodiment, the tube is formed by steps including providing a container, providing a formulation comprising mannitol, boric acid, sodium formate, sodium borate, water, glutamine, and a surfactant, and disposing the formulation into the container. Optionally, the formulation is lyophilized, or freeze-dried, as is well known in the art. The container is then typically evacuated to an extent that will draw into the tube a particular volume of urine. The amount of formulation is based on this draw volume, such that a desired additive to urine ratio is achieved. Typical ranges for the components of the formulation, in milligrams component per milliliter of the draw volume of urine plus any volume provided by the formulation, is about 2 to about 5 mannitol, about 2 to about 5 boric acid, about 1 to about 4 sodium formate, and about 1 to about 4 sodium borate. Typical amounts of glutamine are about 0.1 to about 0.2.

The formulation was found to be advantageous when lyophilized in a plastic tube, typically a PET (polyethylene terephthalate) tube, by providing excellent shelf life in maintaining preservative integrity in terms of preventing visual discoloration or insolubility due to moisture absorption or “melt-back” often seen in lyophilized additives in plastic containers.

### DETAILED DESCRIPTION OF THE INVENTION

According to the invention, any container may be used for urine collection. Typically, collection takes place in an evacuated tube, more typically a plastic tube formed from a material such as PET. Other plastic materials are also possible. Evacuated tubes are well known in the art, and are widely used, for example, in blood collection.

In one embodiment, a urine collection tube is formed as follows. (The process for a single tube is described, but this would typically be done for a large number of tubes.) The stabilizing formulation is mixed. This bulk formulation typically contains mannitol, boric acid, sodium formate, sodium borate, water, glutamine, and a surfactant (typically non-ionic). Amounts of each additive are discussed below. A typical surfactant is Tween® 80, but other surfactants known in the art are also suitable. This bulk formulation is then disposed into the tubes, e.g., through conventional valve mechanisms. The amount of dispense, based on the desired ratios discussed below, is controlled by the valves. Some amount of complexing of the individual additives is to be expected.

The tubes are then typically placed into an evacuation chamber, where pressure is lowered to a level that will provide the desired draw into the tube. It is possible, however, for the tube to remain non-evacuated, in which case other transfer techniques for getting the urine into the tube (i.e., unaided by vacuum) would be required. It is possible to first lyophilize, or freeze-dry, the formulation, either separately from, but more typically within, the evacuation chamber. Lyophilization is well-known in the art of specimen collection containers, as well as in other arts such as food stabilization.

In the case of an evacuation chamber, once the pressure reaches the desired level, closures, typically including an air-tight stopper to maintain vacuum, is placed onto the tube. For evacuated tubes, the closure will have a pierceable septum, which allows the tube to be placed over a cannula in fluid connection with a urine reservoir, such that the urine will be drawn in by the vacuum.

Regardless of whether the tube is evacuated, it is sterilized, e.g., by exposure to Cobalt 60 radiation as is known in the art. Once sterilized, the tube is ready for use.

The mannitol, sodium borate, and boric acid are the primary components responsible for maintaining the microbial system at or near its original condition, e.g., maintaining preservation of systems containing streptococcus faecalis and pseudomonas aeruginosa. Glutamine is effective in maintaining Pseudomonas species. The sodium formate generally provides a buffering function. The surfactant, as expected, facilitates processing and dispersing of the formulation.

The appropriate amounts of the individual components of the formulation are generally based on the effective amount of component per mL of urine to be drawn or dispensed into the tube (referred to hereafter as the “draw volume”). In other words, the container of the invention is designed to ensure that sufficient formulation interacts with the urine to provided the desired stabilization. Useful ranges have been found to include, in milligrams of component per milliliter of the draw volume of urine plus any volume provided by the formulation, about 2 to about 5 mannitol, about 2 to about 5 boric acid, about 1 to about 4 sodium formate, and about 1 to about 4 sodium borate. The amount of glutamine (typically L-glutamine) is about 0.1 to about 0.2.

A particularly useful formulation contains, again in milligrams per milliliter of the draw volume of urine plus any volume provided by the formulation, about 2 to about 5 mannitol, about 2 to about 4 boric acid, about 1 to about 3 sodium formate, about 1 to about 4 sodium borate, and about 0.1 to 0.2 glutamine.

In the bulk formulation, i.e., the aqueous solution dispensed into the tube, water is generally present in an amount ranging from about 0.75 to about 0.85 mL water per mL of overall bulk formulation. However, a wide variety in water content is possible, depending on the solubility of the additives, and the particular process being employed. Generally, the minimum amount of water sufficient to ensure solubility is used, since any excess water would only dilute collected urine, or lengthen a lyophilization process.

A variety of surfactants are possible, with non-ionic surfactants being the most useful. In the case of Tween 80 (generally a 1% solution thereof), a polyoxyethylene sorbitan monoleate, a relatively small amount is sufficient in the bulk formulation, for example in the area of hundredths of a mL per mL of the overall bulk formulation. Other surfactants that may be useful include polysorbate, amphoteric compounds containing carboxylate or phosphate groups, or non-ionic compounds such as fatty acid esters, propylene glycol, sorbitan, or sucrose.

In use, the tube (when evacuated) is typically used with a urine collection container having a cap and a urine reservoir. One type of cap contains a sheathed cannula in fluid connection with the urine reservoir. The pierceable septum on the tube closure is placed over the cannula, and urine is drawn through the cannula into the tube by the vacuum. Alternatively, where the cap has no such features, a transfer device may be used, the device having a straw at one end for placing into the urine reservoir, and at the opposite end a tube holder with a sheathed cannula in fluid communication with the straw. The tube is placed into the holder and over the cannula, to draw urine through the straw and into the tube. Other techniques are also possible, e.g., manual transfer, such as by pipette, from a urine reservoir into a non-evacuated tube.

Other embodiments of the invention will be apparent to those skilled in the art from consideration of the specification and practice of the invention disclosed herein.

What is claimed is:

1. A process for fabricating a container for collection and stabilization of a urine sample, comprising the steps of:
providing a container,
providing a formulation comprising mannitol, boric acid, sodium formate, sodium borate, water, glutamine, and a surfactant, and
disposing the formulation into the container.

2. The process of claim 1, further comprising the step of lyophilizing the formulation subsequent to disposing the formulation into the container.

3. The process of claim 2, further comprising the step of at least partially evacuating the container.

4. The process of claim 1, wherein the container is formed from plastic.

5. The process of claim 4, wherein the plastic is polyethylene terephthalate.

6. The process of claim 1, wherein the formulation consists essentially of mannitol, boric acid, sodium formate, sodium borate, water, glutamine, and a surfactant.

7. The process of claim 1, wherein the container is a tube having a closure thereon, the closure having a pierceable septum.

8. A process for fabricating a container for collection and stabilization of a urine sample, comprising the steps of:
providing a container,
providing a formulation comprising mannitol, boric acid, sodium formate, and sodium borate,
disposing the formulation into the container, and
evacuating the container to provide for an approximate draw volume of urine, wherein the formulation comprises, in milligrams per milliliter of the draw volume of urine plus any volume provided by the formulation, about 2 to about 5 mannitol, about 2 to about 5 boric acid, about 1 to about 4 sodium formate, and about 1 to about 4 sodium borate.

9. The process of claim 8, wherein the formulation further comprises glutamine and a surfactant.

10. The process of claim 9, wherein the formulation comprises about 0.1 to about 0.2 glutamine.

11. The process of claim 8, further comprising the step of lyophilizing the formulation.

12. The process of claim 8, wherein the container is formed of plastic.

13. The process of claim 12, wherein the plastic is polyethylene terephthalate.

14. The process of claim 8, wherein the container is a tube having a closure thereon, the closure having a pierceable septum.

15. A process for fabricating a container for collection and stabilization of a urine sample, comprising the steps of:
providing a container,
providing a formulation comprising mannitol, boric acid, sodium formate, and sodium borate,
disposing the formulation into the container, and
evacuating the container to provide for an approximate draw volume of urine, wherein the formulation comprises, in milligrams per milliliter of the draw volume of urine plus any volume provided by the formulation, about 2 to about 5 mannitol, about 2 to about 4 boric acid, about 1 to about 3 sodium formate, and about 1 to about 4 sodium borate.
